(19) United States
(12) Patent Application Publication
(10) Pub. No.: US 2026/0060630 A1
(43) Pub. Date: Mar. 5, 2026

(54) METHOD AND APPARATUS FOR VISUALIZING CROSS-SECTIONAL SIZE OF ROOT CANAL

(71) Applicants: Ewoosoft Co., Ltd., Gyeonggi-do (KR); VATECH EWOO Holdings Co., Ltd., Gyeonggi-do (KR)

(72) Inventors: Sung Ho KIM, Gyeonggi-do (KR); Seo Yeon PARK, Gyeonggi-do (KR); Min Kyung CUHN, Gyeonggi-do (KR)

(73) Assignees: Ewoosoft Co., Ltd., Gyeonggi-do (KR); VATECH EWOO Holdings Co., Ltd., Gyeonggi-do (KR)

(21) Appl. No.: 19/309,590

(22) Filed: Aug. 25, 2025

(30) Foreign Application Priority Data
Aug. 27, 2024 (KR) 10-2024-0114799

(51) Int. Cl. A61B 6/51; A61B 6/00; A61B 6/03; A61B 6/46; G06T 15/20

(52) U.S. Cl. CPC A61B 6/51; A61B 6/032; A61B 6/465; A61B 6/466; A61B 6/469; A61B 6/5217; G06T 15/205; G06T 2207/10081; G06T 2207/30036

(57) ABSTRACT

Proposed are a method and an apparatus for visualizing a cross-sectional size of the root canal, the method being performed by at least one processor. The method may include displaying an image of a tooth by using CT image data of at least one tooth and three-dimensional rendering data of the root canal of the tooth, calculating, in response to a user selecting a target region of interest by using the image of the tooth, a cross-sectional size of at least a portion of the root canal included in the target region of interest, and displaying at least a portion of the calculated cross-sectional size by a visual factor.

### CROSS REFERENCE TO RELATED APPLICATION

The present application claims priority to Korean Patent Application No. 10-2024-0114799, filed Aug. 27, 2024, the entire contents of which are incorporated herein for all purposes by this reference.

### BACKGROUND

### Technical Field

The present disclosure relates to an image processing technology for dental image processing. More particularly, the present disclosure relates to a method and an apparatus for calculating and visualizing a cross-sectional size of the root canal by using dental image data acquired through Computed Tomography (CT).

### Description of the Related Art

In the dental medical field, when a tooth is damaged due to severe caries, a nerve treatment is performed so as to remove a damaged dental pulp tissue. The damaged dental pulp tissue inside the root canal of the tooth is removed by using a tool such as a file for the nerve treatment. Since the thickness of the file is small, there is a risk that the file is broken due to severe stress applied to the file when the root canal of the tooth to be treated has a large curvature. When the file is broken inside the root canal, it is difficult to remove the file, and this situation may cause secondary inflammation if a portion of the file remains inside the root canal.

Therefore, in order to successfully perform the nerve treatment for the tooth, the treatment is required to be performed so that the file is not broken. To this end, the length, the thickness, and the curvature of the root canal of the tooth is required to be identified in advance. Conventionally, for identifying the length, the thickness, and so on of the root canal of the tooth, the root canal was displayed in a single two-dimensional image, the root canal was roughly identified by the naked eyes, and then the thickness and so on of the root canal was estimated. However, according to this method, since the shape of the root canal is roughly identified in the single two-dimensional image, the three-dimensional shape of the root canal through which the file passes cannot be properly identified.

### SUMMARY

Accordingly, the present disclosure has been made keeping in mind the above problems occurring in the related art, and an objective of the present disclosure is to provide a method and an apparatus for visualizing a cross-sectional size of the root canal.

The present disclosure may be implemented in various manners such as a method, an apparatus, a computer program stored in a readable storage medium, and so on.

According to an aspect of the present disclosure, there is provided a method for visualizing a cross-sectional size of the root canal, the method being performed by at least one processor, and the method including: displaying an image of a tooth by using CT image data of at least one tooth and three-dimensional rendering data of the root canal of the tooth; calculating, in response to a user selecting a target region of interest by using the image of the tooth, a cross-sectional size of at least a portion of the root canal included in the target region of interest; and displaying at least a portion of the calculated cross-sectional size by a visual factor.

According to another aspect of the present disclosure, a computer program stored in a computer-readable recording medium may be provided so as to perform the method for visualizing the cross-sectional size of the root canal on a computer.

According to still another aspect of the present disclosure, there is provided an apparatus for visualizing a cross-sectional size of the root canal, the apparatus including: a storage unit configured to store CT image data of at least one tooth and three-dimensional rendering data of the root canal of the tooth; and an image processing unit configured to display an image of the tooth by using the CT image data of at least one tooth and the three-dimensional rendering data of the root canal of the tooth, the image processing unit being configured to calculate, in response to a user selecting a target region of interest by using the image of the tooth, a cross-sectional size of at least a portion of the root canal included in the target region of interest, and the image processing unit being configured to display at least a portion of the calculated cross-sectional size by a visual factor.

According to aspects of the present disclosure, by visualizing the target region of interest and the cross-sectional size of the root canal by using the CT image of the tooth and the three-dimensional rendering data of the root canal, a nerve treatment for removing a damaged dental pulp tissue in the root canal may be efficiently performed.

According to aspects of the present disclosure, by using various visual factors to output the cross-sectional size of the root canal, a path through which a tool such as a file used in the nerve treatment of the root canal passes may be appropriately guided.

According to aspects of the present disclosure, by using various visual factors to output the cross-sectional size of the root canal, side effects such as secondary inflammation due to damage of the file used in the nerve treatment of the root canal may be prevented from occurring.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a block diagram illustrating a configuration of an apparatus for visualizing a cross-sectional size of the root canal according to an embodiment of the present disclosure;

FIG. 2 is a flowchart illustrating an example of a method for visualizing a cross-sectional size of the root canal according to an embodiment of the present disclosure;

FIG. 3 is a photograph showing a CT image of the teeth and an image of three-dimensional rendering data of the root canal according to an embodiment of the present disclosure;

FIG. 4 shows photographs showing the method for visualizing the cross-sectional size of the root canal according to an embodiment of the present disclosure;

FIG. 5 is a photograph showing an image illustrating the method for visualizing the cross-sectional size of the root canal according to another embodiment of the present disclosure;

FIG. 6 is a flowchart illustrating an example of a method for calculating a cross-sectional size of the root canal according to an embodiment of the present disclosure;

FIG. 7A shows photographs showing images illustrating a method for calculating a centerline of the root canal in a target region of interest selected by a user according to an embodiment of the present disclosure; and

FIG. 7B shows photographs showing images illustrating a method for calculating a circle or a sphere that is inscribed in a reference surface of the centerline of the root canal according to an embodiment of the present disclosure.

### DETAILED DESCRIPTION

Hereinafter, an embodiment of the present disclosure will be described in detail with reference to the accompanying drawings.

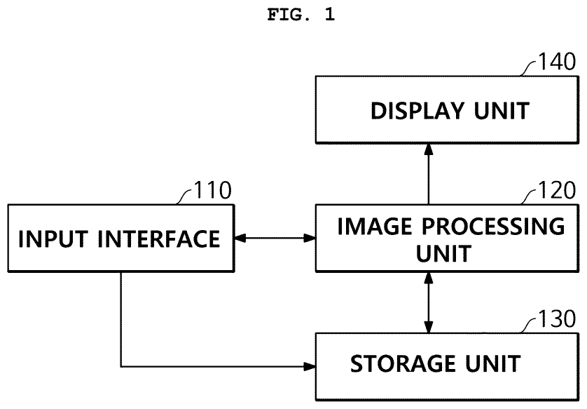

FIG. 1 is a block diagram illustrating a configuration of an apparatus for visualizing a cross-sectional size of the root canal according to an embodiment of the present disclosure.

As illustrated in FIG. 1, an apparatus 100 for visualizing a cross-sectional size of the root canal may include an input interface 110, an image processing unit 120, a storage unit 130, and a display unit 140.

The input interface 110 may be formed of hardware and software modules for inputting a user command in order to perform image processing according to various embodiments of the present disclosure. The input interface 110 may be used to input various necessary commands to the image processing unit 120, may be used to input various image data such as CT image data of at least one tooth acquired through CT scanning, or may be used to receive a user input for performing various image processing for part or all of a displayed image. The input interface 110 may also be used to specify and input an arbitrary point in a dental CT image or a panoramic image.

In an embodiment, the input interface 110 may include a keyboard, a keypad, a touch-pad, a mouse, and so on of a computer, but the type of the input interface is not limited thereto. For example, the input interface 110 may include a Graphical User Interface (GUI) that is capable of being controlled by using the input devices described above. Such a GUI may include a means for implementing a navigator including an upper limit line, a lower limit line, and a reference line on a screen. The display unit 140 is configured to display various images according to various embodiments of the present disclosure, and may include various display devices such as an LCD display, an LED display, an AMOLED display, a CRT display, and so on.

The storage unit 130 may be used to store data of various images such as a CT image of at least one tooth acquired through CT scanning. The storage unit 130 may be used to store image data of intermediate results acquired by performing image processing according to various embodiments of the present disclosure, image data of final results acquired by performing image processing according to various embodiments of the present disclosure, and values of variables required to perform image processing according to various embodiments of the present disclosure.

In various embodiments, the storage unit 130 may store the aforementioned various images in a Digital Imaging and Communications in Medicine (DICOM) format or in a general image file format (BMP, JPEG, TIFF, and so on). The storage unit 130 may further store software and/or firmware required for implementation of the image processing unit 120. The storage unit 130 may be implemented as at least one storage medium selected from a flash memory, a hard disk, a multimedia card (MMC), a card-type memory (for example, a Secure Digital (SD) card, an extreme digital (XD) card, and so on), a Random Access Memory (RAM), a Static Random Access Memory (SRAM), a Read-Only Memory (ROM), an Electrically Erasable Programmable Read-Only Memory (EEPROM), a Programmable Read-Only Memory (PROM), a magnetic memory, a magnetic disk, and an optical disk. However, those skilled in the art will appreciate that the implementation form of the storage unit 130 is not limited thereto.

The image processing unit 120 may be configured to display a CT image of at least one tooth on the display unit 140 by using CT image data of at least one tooth. In an embodiment, the image processing unit 120 may be further configured to generate three-dimensional rendering data of the root canal of at least one tooth by using CT image data and to display the three-dimensional rendering data on the display unit 140. The image processing unit 120 may be configured to calculate a cross-sectional size of at least a portion of the root canal included in a target region of interest, in response to a user selecting a target region of interest among at least one tooth or the root canal included in the CT image data and/or the three-dimensional rendering data.

In an embodiment, selecting the target region of interest among at least one tooth or the root canal included in the CT image data and/or the three-dimensional rendering data may be realized by the user selecting a start point and/or an end point of the root canal through the input interface 110 or by placing the navigator on at least one tooth or the root canal included in the CT image data and/or the three-dimensional rendering data.

In an embodiment, the image processing unit 120 may further be configured to use the CT image data so as to calculate three-dimensional coordinates of the root canal by using an edge detection algorithm, an image segmentation algorithm, and so on as an example, and may further be configured to render at least a portion of the root canal in a three-dimensional form on the basis of the calculated three-dimensional coordinates.

In an embodiment, the image processing unit 120 may be further configured to calculate a cross-sectional size of at least a portion of the root canal included in the target region of interest in response to the user selecting the target region of interest among the displayed images of the teeth through the input interface 110, the displayed images using the CT image data for the teeth and the three-dimensional rendering data of the root canal of the teeth. To this end, the image processing unit 120 may calculate a path of at least a portion of the root canal included in the target region of interest, and may calculate an inscribed circle of the root canal on the calculated path. In addition, in response to the user selecting a specific position of the calculated path through the input interface 110, the image processing unit 120 may calculate an inscribed circle of the root canal at a specific position. Alternatively, the image processing unit 120 may calculate a plurality of inscribed circles of the root canal at a predetermined interval on the calculated path.

In an embodiment, the image processing unit 120 may further be configured to display at least a portion of the calculated cross-sectional size of the root canal by a visual factor. To this end, the image processing unit 120 may display the calculated cross-sectional size in the form of a circle that is inscribed in at least a portion of a cross-section of the root canal. For example, the image processing unit 120 may display the calculated cross-sectional size in the form of a circle overlapping with the three-dimensional rendering data of the root canal. In another example, the image processing unit 120 may display the calculated cross-sectional size in the form of a circle overlapping with the CT image data of the root canal. Alternatively or additionally, the image processing unit 120 may display the calculated cross-sectional size in the form of a sphere that is inscribed in at least a portion of the root canal. For example, the image processing unit 120 may display the calculated cross-sectional size in the form of a sphere overlapping with the three-dimensional rendering data of the root canal. Alternatively or additionally, the image processing unit 120 may display the calculated cross-sectional size by visual factors different from each other according to the corresponding cross-sectional sizes. For example, the image processing unit 120 may display a three-dimensional shape having a size, a color, or a thickness different according to the calculated cross-sectional size.

According to the image processing unit 120 having the configuration described above, by visualizing the region of interest and the cross-sectional size of the root canal by using the CT image of the teeth and the three-dimensional rendering data of the root canal, a nerve treatment for removing a damaged dental pulp tissue in the root canal may be efficiently performed. In addition, by outputting the cross-sectional size of the root canal by using various visual factors, the image processing unit 120 may properly guide a path through which a tool such as a file used for performing a nerve treatment of the root canal passes. In addition, since the image processing unit 120 outputs the cross-sectional size of the root canal by using various visual factors, side effects such as secondary inflammation due to damage of the file used in the nerve treatment of the root canal may be prevented from occurring.

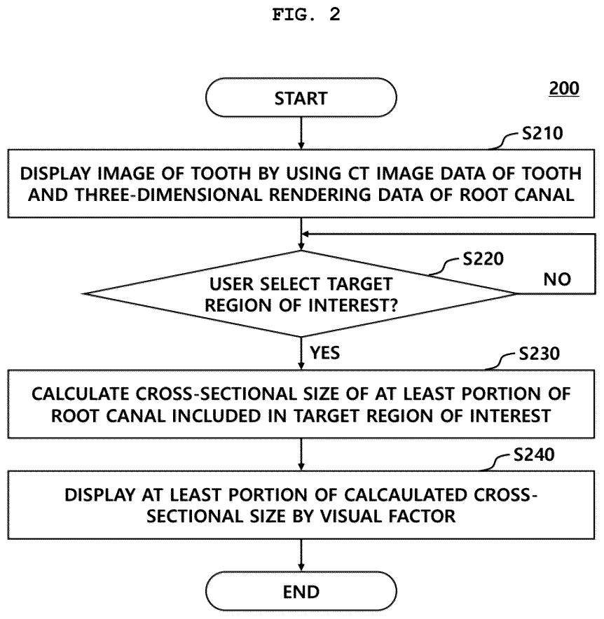

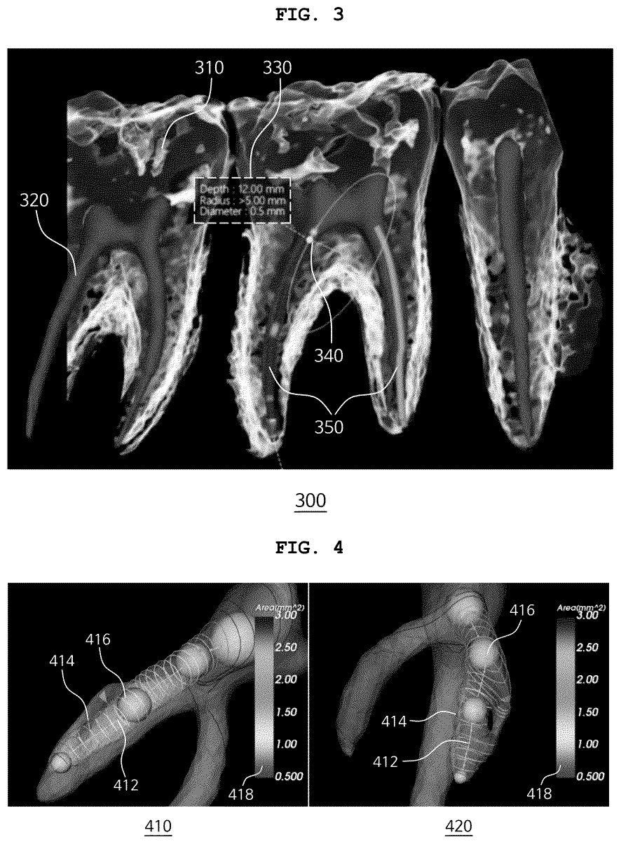

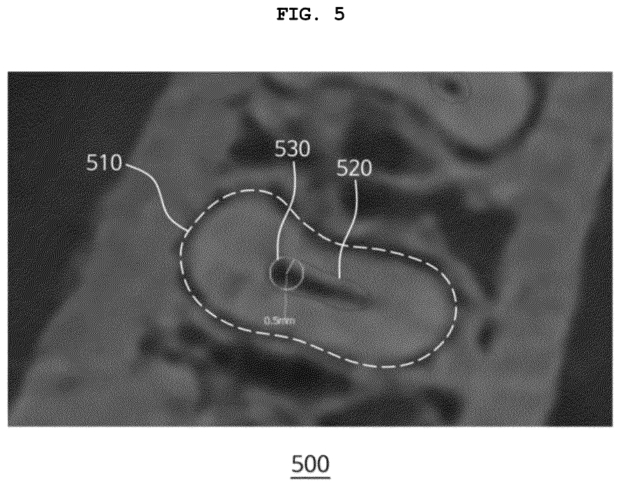

FIG. 2 is a flowchart illustrating an example of a method for visualizing a cross-sectional size of the root canal according to an embodiment of the present disclosure. FIG. 3 is a photograph showing a CT image of the teeth and an image of three-dimensional rendering data of the root canal according to an embodiment of the present disclosure. FIG. 4 shows photographs showing the method for visualizing the cross-sectional size of the root canal according to an embodiment of the present disclosure. FIG. 5 is a photograph showing an image illustrating the method for visualizing the cross-sectional size of the root canal according to another embodiment of the present disclosure.

In an embodiment, a method 200 for visualizing a cross-sectional size of the root canal may be performed by at least one processor. For example, the processor may be a processor that implements at least one selected from the input interface 110, the image processing unit 120, the storage unit 130, and the display unit 140 in FIG. 1.

The method 200 may begin with a process S210 in which the processor displays an image of the tooth by using CT image data of at least one tooth and three-dimensional rendering data of the root canal of the tooth.

In an embodiment, the processor may be configured to use the CT image data of the tooth so as to calculate three-dimensional coordinates of the root canal by using the edge detection algorithm, the image segmentation algorithm, and so on as an example, and may be configured to render at least a portion of the root canal in the three-dimensional form on the basis of the calculated three-dimensional coordinates. For example, referring to a tooth image 300 illustrated in FIG. 3, the processor may calculate the three-dimensional coordinates of the root canal from a CT image data 310 of the tooth, and may calculate a three-dimensional rendering data 320 that represents at least a portion of the root canal on the basis of the calculated three-dimensional coordinates. The processor may display the three-dimensional rendering data 320 of the root canal by overlapping the three-dimensional rendering data 320 of the root canal on a position of the root canal on the CT image data 310 of the tooth through a display unit (for example, the display unit 140 in FIG. 1).

In addition, in response to the user selecting a target region of interest by using the image of the tooth, the processor may calculate a cross-sectional size of at least a portion of the root canal included in the target region of interest S220 and S230.

For example, referring to FIG. 3, the user may select a target region of interest that includes a specific position 340 with respect to the three-dimensional rendering data of the root canal in the image 300 of the teeth displayed on the display unit through the input interface. In an embodiment, selecting the target region of interest may be realized by the user selecting a start point and/or an end point of the root canal through the input interface or by placing the navigator on at least one tooth or the root canal included in the CT image data and/or the three-dimensional rendering data.

In an embodiment, calculating a cross-sectional size of at least a portion of the root canal included in the target region of interest may include calculating a path of at least a portion of the root canal included in the target region of interest and then calculating an inscribed circle of the root canal on the calculated path. In addition, calculating the cross-sectional size of at least a portion of the root canal included in the target region of interest may include calculating an inscribed circle of the root canal at a specific position in response to the user selecting a specific position of the calculated path through the input interface. For example, as illustrated in FIG. 3, in response to the user selecting the specific position 340 of a calculated path 350 through the input interface, after calculating an inscribed circle of the root canal at the specific position 340, information related to the inscribed circle may be displayed (for example, a depth (12.00 mm) on the path, a radius of the inscribed circle (equal to or less than 5.00 mm), a diameter of the inscribed circle (0.5 mm), and so on). Alternatively, calculating the cross-sectional size of at least a portion of the root canal included in the target region of interest may include calculating a plurality of inscribed circles of the root canal at a predetermined interval on the calculated path.

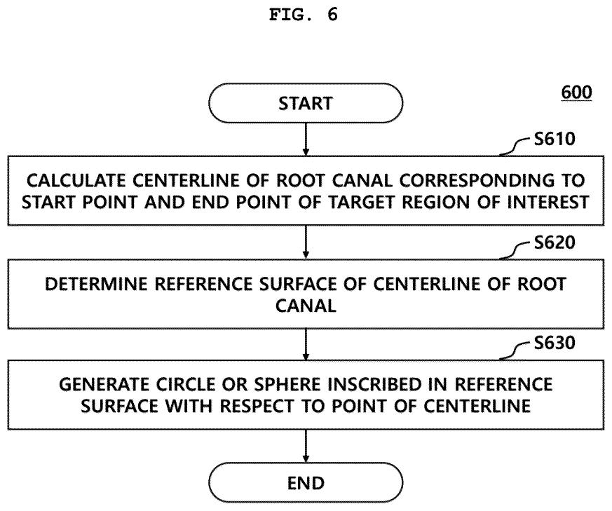

In an embodiment, calculating the cross-sectional size of at least a portion of the root canal included in the target region of interest may include determining a centerline of the root canal within the three-dimensional rendering data or a three-dimensional model of the root canal, determining a circle or a sphere that is inscribed in a reference surface of the corresponding centerline, and then determining a radius or a diameter of the determined circle or the determined sphere as the cross-sectional size. Hereinafter, in FIG. 6, a method for calculating a cross-sectional size of the root canal will be described in detail.

Then, the processor may display at least a portion of the calculated cross-sectional size by a visual factor S240.

In an embodiment, the processor may display the calculated cross-sectional size in the shape of a circle that is inscribed in at least a portion of a cross-section of the root canal. For example, referring to the tooth images 410 and 420 in FIG. 4, the processor may display the calculated cross-sectional size in the form of a circle 414 that overlaps with the three-dimensional rendering data of the root canal. In another example, referring to a tooth image 500 in FIG. 5, the processor may display the calculated cross-sectional size in the form of a circle 530 overlapping with the CT image data of the root canal 520.

Alternatively or additionally, the processor may display the calculated cross-sectional size in the form of a sphere inscribed in at least a portion of the root canal. For example, referring to the tooth images 410 and 420 in FIG. 4, the processor may display the calculated cross-sectional size in the form of a sphere 416 that overlaps with the three-dimensional rendering data of the root canal.

Alternatively or additionally, the processor may display the calculated cross-sectional size by visual factors that are different from each other according to the corresponding cross-sectional sizes. For example, referring to the tooth images 410 and 420 in FIG. 4, the processor may display two-dimensional or three-dimensional shapes having a size, a color, or a thickness different according to the calculated cross-sectional size. As illustrated in FIG. 4, in the tooth images 410 and 420, a color index 418 according to an area of a cross-section in the unit of mm² may be displayed for the calculated cross-sectional sizes, and an inscribed circle 414 or an inscribed sphere 416 displayed in colors different according to the corresponding color index 418 may be displayed.

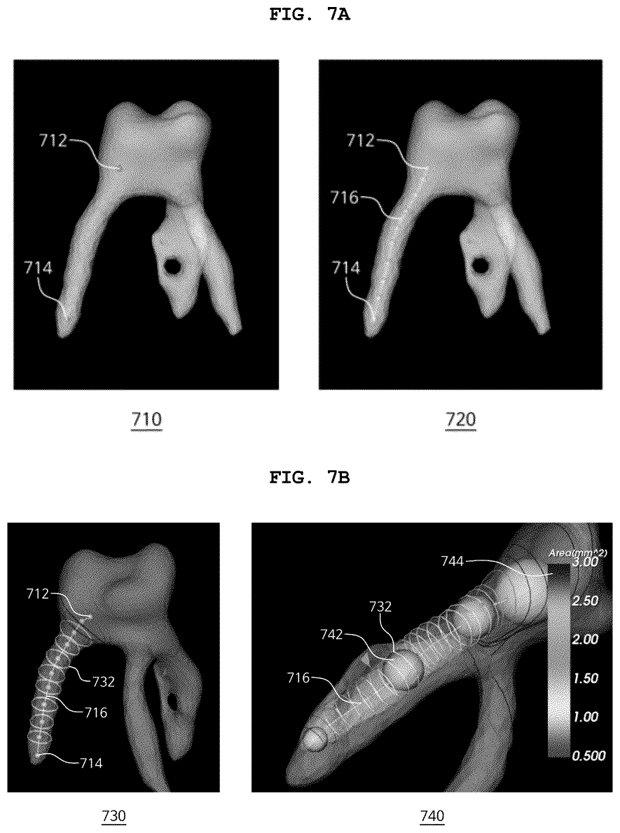

FIG. 6 is a flowchart illustrating an example of the method for calculating the cross-sectional size of the root canal according to an embodiment of the present disclosure. FIG. 7A shows photographs showing images illustrating a method for calculating a centerline of the root canal in a target region of interest selected by the user according to an embodiment of the present disclosure. FIG. 7B shows photographs showing images illustrating a method for calculating a circle or a sphere that is inscribed in a reference surface of the centerline of the root canal according to an embodiment of the present disclosure.

In an embodiment, a method 600 for calculating the cross-sectional size of the root canal may be performed by at least one processor. For example, the processor may be a processor that implements at least one selected from the input interface 110, the image processing unit 120, the storage unit 130, and the display unit 140 in FIG. 1. In addition, the method 600 may correspond to the process S230 in FIG. 2.

The method 600 may begin with a process S610 in which the processor calculates a centerline of the root canal corresponding to a start point and an end point of the target region of interest. For example, referring to tooth images 710 and 720 in FIG. 7A, a centerline 716 of the root canal corresponding to a start point 712 and an end point 714 of the target region of interest selected by the user may be calculated in the tooth image 720.

In addition, the processor may determine a reference surface of the centerline of the root canal S620. For example, referring to a tooth image 730 in FIG. 7B, a reference surface 732 at a specific point on the centerline 716 of the root canal may be defined as an inscribed circle perpendicular to the centerline 716 at that point or a cross-section of a three-dimensional model.

Then, the processor may generate a circle or a sphere that is inscribed in the reference surface with respect to the point of the centerline S630. A size of the cross-section or a radius of the inscribed circle or the inscribed sphere may be determined as the size of a cross-section of the root canal. For example, referring to the tooth image 740 in FIG. 7B, a sphere 742 inscribed in the reference surface 732 with respect to the point of the centerline 716 may be generated and displayed. As described above, a cross-section with respect to the center point of the inscribed sphere 742 may be determined as the inscribed circle. In addition, in the tooth image 740, a color index 744 according to an area of a cross-section in the unit of mm² may be displayed for the calculated cross-sectional sizes, and an inscribed circle or the inscribed sphere 742 displayed in colors different according to the corresponding color index 744 may be displayed.

In an embodiment, the centerline 716 of the root canal may be defined as a weighted shortest path that is tracked between the start point 712 and the end point 714. Specifically, the corresponding path may be limited to be placed on the Voronoi diagram of the three-dimensional model (or the three-dimensional rendering data) of the root canal. Here, the Voronoi diagram may indicate positions of centers of the maximal inscribed spheres within the three-dimensional model of the root canal. That is, for each point belonging to the Voronoi diagram, there may be maximal inscribed spheres centered on the corresponding point. The centerline 716 of the root canal is determined on the paths defined in the Voronoi diagram of the three-dimensional model of the root canal as described above, and an integral value of the radius of the maximal inscribed spheres on the corresponding paths may be minimized. This is the same as finding a minimum path in a radius metric method. Specifically, this method may be implemented by starting propagation from the start point (i.e., the starting point of the centerline) by using an inverse of radius as a wave speed, recording a propagation arrival time at all points in the Voronoi diagram, and then backtracking the centerline from the end point (i.e., the end point of the centerline) along a gradient of an arrival time. Each point on the centerline defined in the Voronoi diagram configured as described above may be related to the corresponding maximal inscribed sphere.

What is claimed is:

1. A method for visualizing a cross-sectional size of a root canal, the method being performed by at least one processor, and the method comprising:
displaying an image of a tooth by using CT image data of at least one tooth and three-dimensional rendering data of the root canal of the tooth;
calculating, in response to a user selecting a target region of interest by using the image of the tooth, a cross-sectional size of at least a portion of the root canal included in the target region of interest; and
displaying at least a portion of the calculated cross-sectional size by a visual factor.

2. The method of claim 1, wherein the calculating of the cross-sectional size of at least a portion of the root canal comprises:
calculating a path of at least a portion of the root canal included in the target region of interest; and
calculating an inscribed circle of the root canal on the calculated path.

3. The method of claim 2, wherein the calculating of the cross-sectional size of at least a portion of the root canal comprises calculating, in response to the user selecting a specific position of the calculated path, an inscribed circle of the root canal at the specific position or calculating a plurality of inscribed circles of the root canal at a predetermined interval on the calculated path.

4. The method of claim 1, wherein the displaying at least a portion of the calculated cross-sectional size by the visual factor comprises displaying the calculated cross-sectional size in a form of a circle or a sphere inscribed in a cross-section of at least a portion of the root canal.

5. The method of claim 4, wherein the displaying of the calculated cross-sectional size in the form of the circle or the sphere inscribed in the cross-section of at least a portion of the root canal comprises displaying the calculated cross-sectional size in a form of a circle or a sphere overlapping with the three-dimensional rendering data of the root canal.

6. The method of claim 4, wherein the displaying of the calculated cross-sectional size in the form of the circle or the sphere inscribed in the cross-section of at least a portion of the root canal comprises displaying the calculated cross-sectional size in a form of a circle overlapping with CT image data of the root canal.

7. The method of claim 1, wherein the displaying at least a portion of the calculated cross-sectional size by the visual factor comprises displaying a two-dimensional shape or a three-dimensional shape having a size, a color, or a thickness different according to the calculated cross-sectional size.

8. An apparatus for visualizing a cross-sectional size of a root canal, the apparatus comprising:
a storage unit configured to store CT image data of at least one tooth and three-dimensional rendering data of the root canal of the tooth; and
an image processing unit configured to display an image of the tooth by using the CT image data of at least one tooth and the three-dimensional rendering data of the root canal of the tooth, the image processing unit being configured to calculate, in response to a user selecting a target region of interest by using the image of the tooth, a cross-sectional size of at least a portion of the root canal included in the target region of interest, and the image processing unit being configured to display at least a portion of the calculated cross-sectional size by a visual factor.
